(19) United States
(12) Patent Application Publication
(10) Pub. No.: US 2023/0134709 A1
(43) Pub. Date: May 4, 2023

(54) INTRAVENOUS BAG WRINGER

(71) Applicant: CareFusion 303, Inc., San Diego, CA (US)

(72) Inventors: Rahul Rajan PUTHUKKAD, Bengaluru (IN); Gene E. MASON, La Habra Heights, CA (US)

(21) Appl. No.: 17/956,105

(22) Filed: Sep. 29, 2022

Related U.S. Application Data
(60) Provisional application No. 63/273,601, filed on Oct. 29, 2021.

(51) Int. Cl. A61M 5/142; A61M 5/152

(52) U.S. Cl. CPC A61M 5/14232; A61M 5/152

(57) ABSTRACT

An IV bag wringer includes a frame defining a frame opening and multiple roller assemblies disposed within the frame opening. Each roller assembly includes a shaft coupled to the frame and a roller coupled to the shaft. Two opposing rollers are positioned to receive an IV fluid bag in a gap between the rollers such that the IV bag wringer hangs on the IV fluid bag. The weight of the IV bag wringer moves it downward along the IV fluid bag so that the opposing rollers squeeze fluid out of the IV fluid bag at a rate greater than a gravity flow rate from the IV fluid bag alone. Hand cranked IV bag wringers, motor actuated IV bag wringers and methods of operating IV bag wringers are also provided.

### CROSS-REFERENCE TO RELATED APPLICATIONS

This application claims the benefit of priority under 35 U.S.C. § 119 to U.S. Provisional Pat. Application Serial No. 63/273,601, entitled “INTRAVENOUS BAG WRINGER,” filed on Oct. 29, 2021, the entirety of which is incorporated herein by reference.

### TECHNICAL FIELD

The present disclosure generally relates to intravenous (IV) sets, in particular to wringers for IV fluid bags.

### BACKGROUND

Blood transfusion is a potential life-saving procedure that can help replace blood lost due to surgery or injury, and also may help if an illness prevents a body from making blood or some blood components correctly. For example, shorter times for conducting blood transfusion are associated with decreased death risk in trauma patients.

Medical treatments often include the infusion of a medical fluid (e.g., blood, plasma) to patients using an IV catheter that is connected though an arrangement of flexible tubing and fittings, commonly referred to as an “IV set,” to a source of fluid, for example, an IV bag. During operation, medical fluid may be required quickly at greatly increased flow rates. Typical IV blood sets use a manual hand pump bulb or flexible container that is squeezed by hand to rapidly increase fluid flow rate, resulting in muscle fatigue.

For these reasons, it is desirable to provide a wringer that mechanically squeezes the IV bag to eliminate muscle fatigue.

### SUMMARY

In one or more embodiments according to the disclosure, an IV bag wringer comprises a frame defining a frame opening; and a plurality of roller assemblies disposed within the frame opening, each roller assembly comprising: a shaft coupled to the frame; and a roller coupled to the shaft, wherein two opposing rollers are positioned to receive an IV fluid bag in a gap between the rollers such that the IV bag wringer hangs on the IV fluid bag, and wherein the IV bag wringer is configured to move downward along the IV fluid bag based on a weight of the IV bag wringer such that the opposing rollers squeeze fluid out of the IV fluid bag at a rate greater than a gravity flow rate from the IV fluid bag alone.

In one or more embodiments according to the disclosure, an IV bag wringer comprises a housing comprising a rear wall, a top wall, a first side wall and an opposing second side wall; a roller assembly coupled to rear wall of the housing, the roller assembly comprising: a first linear slide bearing; a second linear slide bearing; a shaft coupled to the first and second linear slide bearings; and a roller coupled to the shaft; and a crank assembly coupled to the housing and to the first and second linear slide bearings, the crank assembly configured to slidably adjust a position of the first and second linear slide bearings relative to the rear wall, wherein a gap between the roller and the rear wall of the housing is configured to receive an IV fluid bag.

In one or more embodiments according to the disclosure, a method of operating an IV bag wringer comprises coupling an IV set to an IV fluid bag; hanging the IV fluid bag; setting a gravity based fluid flow rate of fluid from the IV fluid bag through the IV set; disposing a top portion of the IV fluid bag within the gap between the two opposing rollers of the IV bag wringer; hanging the IV bag wringer from the IV fluid bag; releasing the IV bag wringer such that a weight of the IV bag wringer moves the two opposing rollers downward along the IV fluid bag at a first speed; and squeezing the IV fluid bag between the two opposing rollers to cause an increased fluid flow rate from the IV fluid bag corresponding to the first speed.

In one or more embodiments according to the disclosure, a method of operating an IV bag wringer comprises coupling an IV set to an IV fluid bag; hanging the IV fluid bag; setting a gravity based fluid flow rate of fluid from the IV fluid bag through the IV set; disposing a top portion of the IV fluid bag within the gap between the roller and the rear wall of the IV bag wringer; rotating a hand crank coupled to the crank assembly; moving the roller downward along the IV fluid bag at a first speed based on a rate of rotation of the hand crank; and squeezing the IV fluid bag between the roller and the rear wall to cause an increased fluid flow rate from the IV fluid bag corresponding to the first speed.

In one or more embodiments according to the disclosure, a method of operating an IV bag wringer comprises coupling an IV set to an IV fluid bag; hanging the IV fluid bag; setting a gravity based fluid flow rate of fluid from the IV fluid bag through the IV set; disposing a top portion of the IV fluid bag within the gap between the roller and the rear wall of the IV bag wringer; actuating a motor coupled to the crank assembly; moving the roller downward along the IV fluid bag at a first speed based on the speed of the motor; and squeezing the IV fluid bag between the roller and the rear wall to cause an increased fluid flow rate from the IV fluid bag corresponding to the first speed.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIG. 2 depicts a top view of a typical assembled IV infusion set with a hand pump and an IV bag.

FIG. 3 depicts a perspective view of an IV bag wringer with an IV bag, according to aspects of the disclosure.

FIG. 4 depicts a top view of the IV bag wringer of FIG. 3, according to aspects of the disclosure.

FIG. 5 depicts a side view of the IV bag wringer of FIG. 3, according to aspects of the disclosure.

FIG. 6 depicts a perspective view of an IV bag wringer with an IV bag, according to aspects of the disclosure.

FIG. 7 depicts a front view of the IV bag wringer of FIG. 6, according to aspects of the disclosure.

FIG. 8 depicts a bottom view of the IV bag wringer of FIG. 6, according to aspects of the disclosure.

FIG. 9 depicts a front view of an IV bag wringer, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

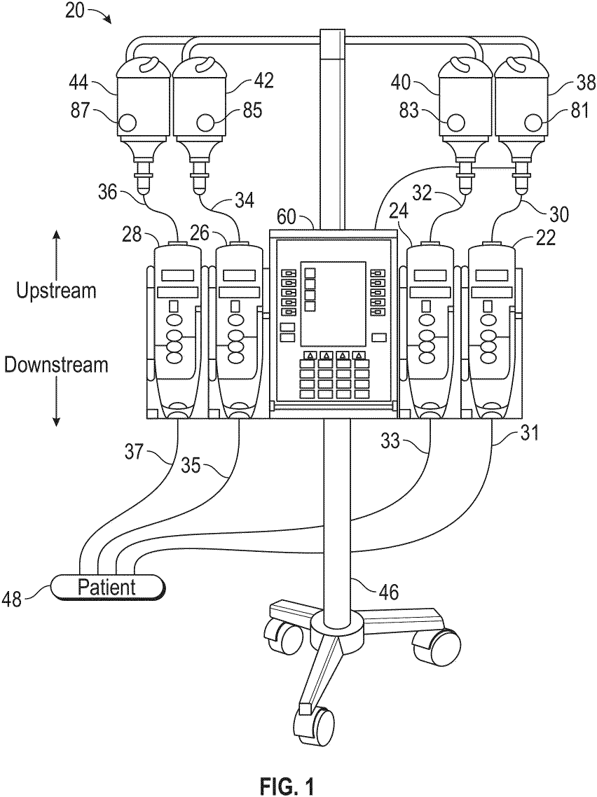

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

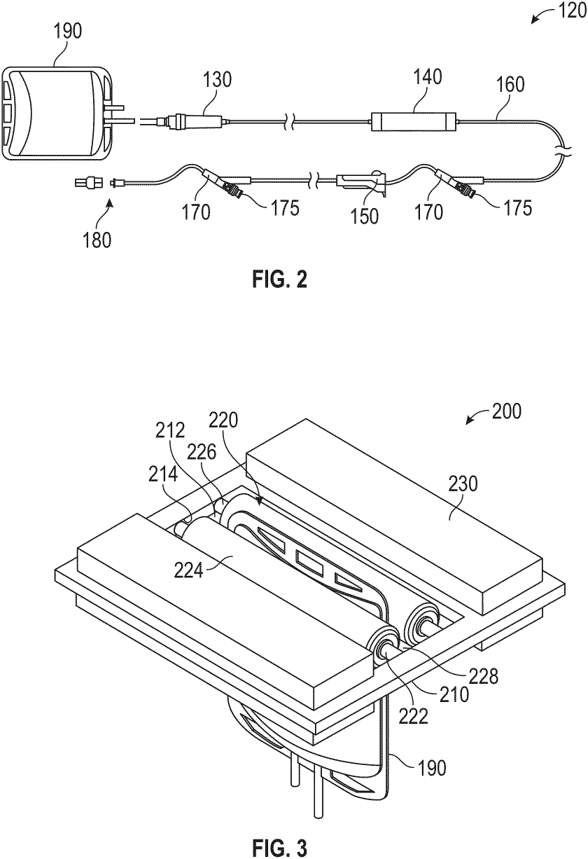

Typically, medical fluid administration sets have more parts than are shown in FIG. 1, such as those shown in FIG. 2. IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2, an IV set 120 may include a drip chamber 130, a hand pump 140 and a roller clamp 150 connected together by tubing 160. The IV set 120 may also include a Y-site 170 having a Y-shaped junction with a needleless connector 175, as well as a luer lock connector 180 at the end of the IV set 120. The luer lock connector 180 may be used for connection to a catheter inserted into a patient, for example. On the other end, the drip chamber 130 may be coupled to an IV bag 190. The IV set 120 may include additional infusion components and may be formed of any combination of components and the tubing 160.

Blood is typically accelerated into a body via a hand-pumped bulb, but this practice results in hand fatigue. In aspects of the disclosure, an IV bag wringer uses one or more rollers to squeeze blood directly from an IV bag. The IV bag wringer may be motorized, hand-cranked or rely on gravity. The motorized and hand cranked IV bag wringers may have a pressure gauge that limits the crank force to the maximum allowed flow rate. The gravity based IV bag wringer may be weighted to exert a force yielding the maximum allowed flow rate.

In aspects of the disclosure, a reusable gravity based IV bag wringer may be coupled to a hanging fluid filled (e.g., blood) IV bag to increase the flow of blood from the IV bag without hand fatigue. Here, the IV bag may be inserted into the IV bag wringer so that the IV bag is positioned between rollers that then squeeze the IV bag as the IV bag wringer is pulled downward by gravity in order to cause a wringing action of the IV bag.

In aspects of the disclosure, hand-cranked reusable mounted IV bag wringer may receive a fluid filled IV bag to increase the flow of blood from the IV bag without hand fatigue. The IV bag wringer cranking speed and corresponding flow rate may be chosen by the clinician. Here, the IV bag may be inserted into the IV bag wringer so that the IV bag is positioned between a roller and a stationary surface that then squeezes the IV bag to cause a wringing action.

In aspects of the disclosure, a motorized reusable mounted IV bag wringer may receive a fluid filled IV bag to increase the flow of blood from the IV bag without hand fatigue. The IV bag wringer frequency and corresponding flow rate may be chosen by the clinician. Here, the IV bag may be inserted into the IV bag wringer so that the IV bag is positioned between a roller and a stationary surface that then squeezes the IV bag to cause a wringing action.

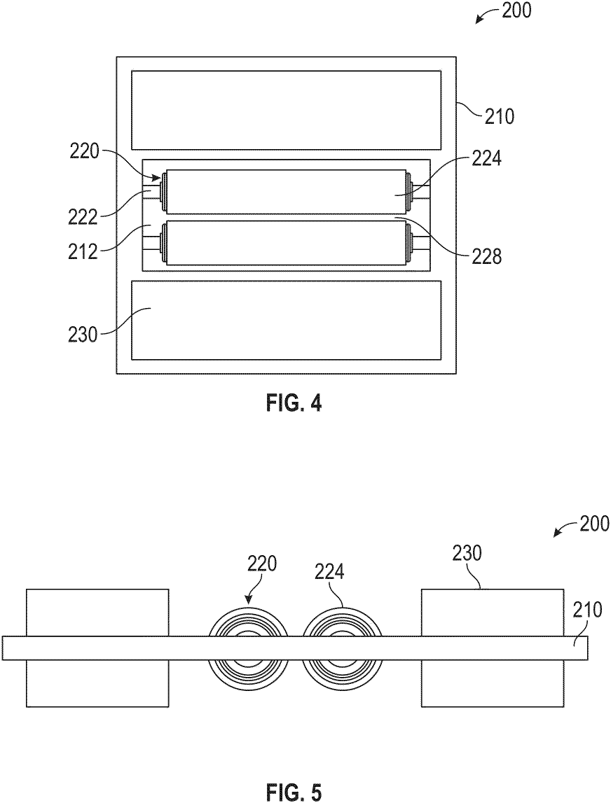

As shown in FIGS. 3 to 5, an IV bag wringer 200 is provided according to aspects of the disclosure. IV bag wringer 200 includes a frame 210 having a frame opening 212 and two roller assemblies 220 coupled to the frame 210, with the roller assemblies 220 disposed within the frame opening 212. Each roller assembly 220 may include a shaft 222 and a roller 224. For example, each end 226 of the shaft 222 may be received by a corresponding cavity 214 in the frame 210. In aspects of the disclosure, the shaft 222 may remain stationary with respect to the frame 210 and the roller 224 may be rotatably mounted to the shaft 222 such that the roller 224 may spin around the shaft 222. In aspects of the disclosure, the shaft 222 may be rotatably coupled to the frame 210 and the roller 224 may be fixedly coupled to the shaft 222 such that the roller 224 and shaft 222 combination rotate together with respect to the frame. For example, the shaft ends 226 may rotate within the cavities 214.

One or more weights 230 may be coupled to the frame 210. In aspects of the disclosure, the weight 230 may be fixedly or removably coupled to the frame 210. For example, the frame 210 and the weights 230 may be integrally formed (e.g., cast, molded, 3D printed). As another example, the frame 210 and the weights 230 may be formed separately and coupled fixedly together (e.g., welded, soldered, bonded, glued). As yet another example, the frame 210 and the weights 230 may be formed separately and coupled removably together (e.g., screwed, bolted, via magnets, via hook and loop material).

In aspects of the disclosure, the weights 230 may be configured to cause the IV bag wringer 200 to move downward along an IV bag 190 based on gravity. Here, the weights 230 may be configured or selected based on a desired flow rate from the IV bag 190. For example, if the flow rate is desired to be somewhat faster than a fully open IV flow rate of the IV set (e.g., IV set 120) without squeezing the IV bag 190 or any component of the IV set 120 (e.g., drip chamber 130, hand pump 140), then the weights 230 may each be selected as a light weight. Similarly, if the flow rate is desired to be very fast in order to empty the IV bag 190 as quickly as possible, then the weights 230 may each be selected as a heavy weight.

Thus, in aspects of the disclosure, if the weights 230 are integral with or fixedly coupled to the frame 210, then a variety of IV bag wringers 200 may be provided so that a user (e.g., healthcare clinician) may choose an appropriately weighted IV bag wringer 200 for the medical issue at hand. Also, in aspects of the disclosure, if the weights 230 are removably coupled to the frame 210, then a variety of weights 230 may be provided so that a user (e.g., healthcare clinician) may choose an appropriate set of weights 230 to quickly couple to a standard IV bag wringer 200 for the medical issue at hand

In use, an IV bag (e.g., IV bag 190) of an IV set (e.g., IV set 120) is inserted between the IV bag wringer 200. For example, the top of the IV bag may be fed in between the rollers 224. The IV set may already be coupled or is then coupled to the IV bag containing a medical fluid (e.g., blood). In an unactuated state, the IV bag wringer 200 may not contact and/or compress the IV bag, thus allowing the blood to flow through the IV set at a rate set by a flow controller (e.g., roller clamp 150). For example, the IV bag wringer 200 may be held in place at the top of the IV bag by a restraining member, such as by being coupled to or suspended from an IV pole (e.g., IV pole 46) or via a removable clip that can be coupled to the bag below the rollers 224. Thus, when it is desired to actuate the IV bag wringer 200, the restraining member may be released (e.g., released from the IV pole coupling) or removed (e.g., the clip removed from the bag).

In aspects of the disclosure, the IV bag wringer 200 may not have a restraint and may be actuated as soon as it is coupled to an IV bag. For example, a clinician may simply feed the IV bag into the rollers 224 and then hang the IV bag from an IV pole, thus allowing the IV bag wringer 200 to be actuated.

In an actuated state, the total weight of the IV bag wringer 200 (e.g., the combined weight of the frame 210, the roller assemblies 220 and the weights 230) causes the IV bag wringer 200 to move down the IV bag due to gravity. This downward movement of the IV bag wringer 200 causes the rollers 224 to rotate, which causes the rollers 224 to compress the IV bag, thus squeezing the IV bag between the two rollers 224 in a steady and/or continuous pumping manner. Thus, the fluid flow rate may be rapidly increased over that of a gravity based flow rate of the IV set without the use of the IV bag wringer 200.

In aspects of the disclosure, the roller assemblies 220 may be positionally adjustable on the frame 210, providing for adjusting the size of the gap 228 between the rollers 224. For example, there may be multiple cavities 214 in the frame 210 such that the shaft ends 226 may be moved between different cavities 214 as needed. As another example, the shaft ends 226 may be disposed within a groove so that they may be slidably moved back and forth as needed. Any suitable coupling may be used between the shafts 222 and the frame 210 (e.g., spring loaded, ratchet, camming). Thus, the IV bag wringer 200 may be adjusted to work with different sized and/or shaped IV bags. In aspects of the disclosure, the IV bag wringer 200 is reusable while the IV bag and/or IV set are disposable. Accordingly, the IV bag wringer 200 may be cleaned as necessary or per medical cleaning protocols.

As shown in FIG. 3, IV bag wringer 200 may be a self-contained device configured to hang directly from an IV bag 190. Accordingly, the IV bag wringer 200 may be small and lightweight so as not to put undue strain on the IV bag 190 or the IV set 120.

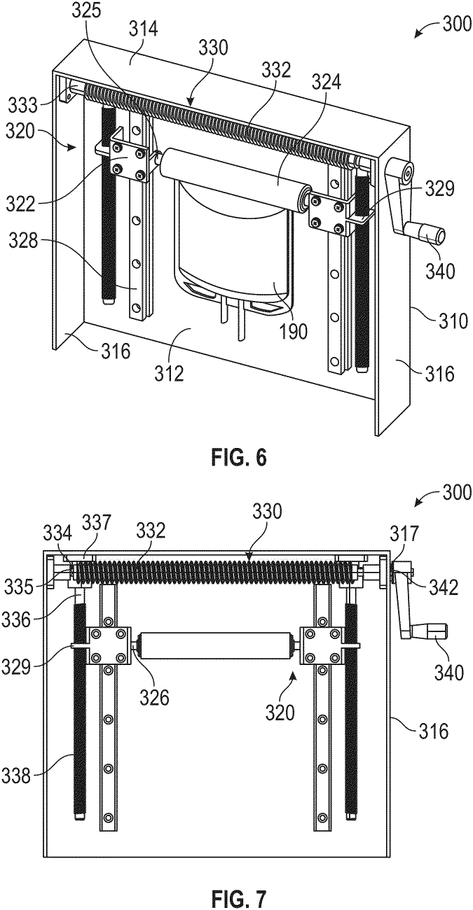

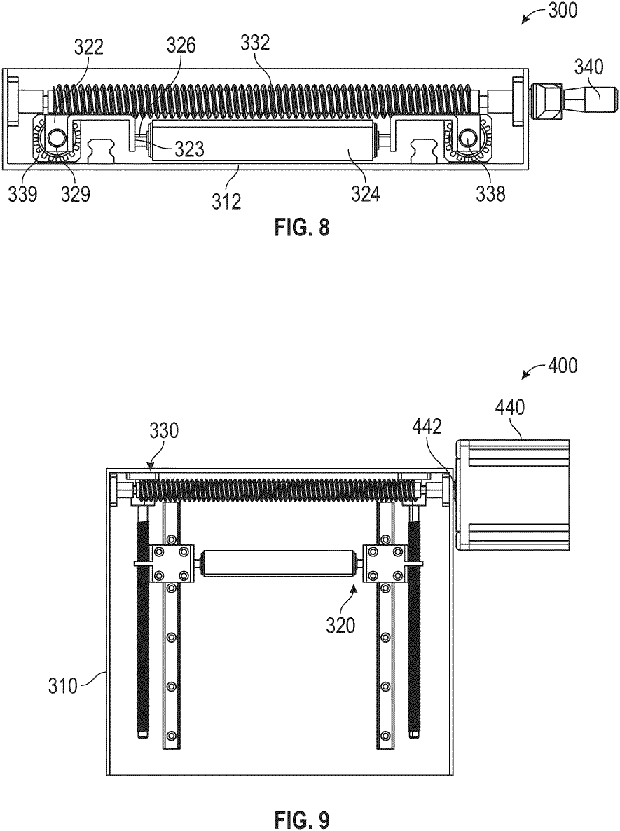

As shown in FIGS. 6 to 8, an IV bag wringer 300 is provided according to aspects of the disclosure. IV bag wringer 300 includes a housing 310 having a rear wall 312, a top wall 314 and two side walls 316. A roller assembly 320 may be coupled to the rear wall 312. The roller assembly 320 may include two linear slide bearings 322 and a roller 324 having a shaft 325. For example, each end 326 of the shaft 325 may be coupled to one of the linear slide bearings 322. In aspects of the disclosure, the shaft 325 may remain stationary with respect to the linear slide bearings 322 and the roller 324 may be rotatably mounted to the shaft 325 such that the roller 324 may spin around the shaft 325. In aspects of the disclosure, the shaft 325 may be rotatably coupled to the linear slide bearings 322 and the roller 324 may be fixedly coupled to the shaft 325 such that the roller 324 and shaft 325 combination rotate together with respect to the linear slide bearings 322. For example, the shaft ends 326 may rotate within cavities 323 of the slide bearings 322.

Each linear slide bearing 322 is movably coupled (e.g., slidable) to a slider guide 328 that is affixed or coupled to the rear wall 312. For example, the slider guide 328 may be a linear rail where the linear slider bearing 322 may slide up and down the rail. The linear slide bearing 322 may also include a threaded opening 329.

IV bag wringer 300 may further include a crank assembly 330. Crank assembly 330 may include a lead screw 332 rotatably coupled to lead screw mounting brackets 333 that are fixedly coupled to the side walls 316. Lead screw ends 334 may be received by and rotate within cavities or openings 335 of the lead screw mounting brackets 333. Side screws 336 may be rotatably coupled to side screw mounting brackets 337 that are fixedly coupled to the top wall 314. The side screw 336 may include a threaded shaft 338 that is received by the threaded opening 329 of the linear slide bearing 322. The side screw 336 may also include a gear 339 that engages/interfaces with the lead screw 332.

IV bag wringer 300 may further include a hand crank 340. Hand crank 340 may be fixedly coupled to one lead screw end 334. For example, a lead screw end 334 may extend through the opening 335 of one lead screw mounting bracket 333, through an opening 317 in the side wall 316 and be received within a cavity or opening 342 of the hand crank 340.

Rotational movement of the hand crank 340 causes the coupled lead screw 332 to rotate, thus causing the engaged gears 339 of the side screws 336 to rotate, thereby causing the threaded shafts 338 to rotate within the threaded openings 329 of the linear slide bearings 322 as well. Depending upon the direction of rotation of the hand crank 340, the linear slide bearings 322 are slidably moved up or down along the slider guides 328 based on the interaction between the threaded shafts 338 rotating within the threaded openings 329 of the linear slide bearings 322. The slidable movement of the linear slide bearings 322 causes the roller 324 to move in the same direction.

In use, as shown in FIG. 6, a fluid containing IV bag 190 may be placed within the IV bag wringer 300 where the IV bag 190 is disposed against the rear wall 312 in between the slider guides 328. For example, the top of the IV bag 190 may be placed or inserted between the rear wall 312 and the roller 324. With the IV bag 190 in position, the hand crank 340 may be operated (e.g., rotated by hand), causing the lead screw 332 to rotate, in turn causing the side screws 336 to rotate. The rotation of the side screws 336 causes the linear slide bearings 322 to move downward along the slider guides 328, in turn causing the roller 324 to move downward. The downward movement of the roller 324 squeezes the IV bag 190 between the rear wall 312 and the roller 324, thus increasing the fluid flow rate out of the IV bag 190 over a gravity based flow rate alone.

At any point where there is still fluid remaining in the IV bag 190, rotation of the hand crank 340 may be stopped and the fluid flow may readjust back to a gravity based flow rate. Similarly, by rotating the hand crank 340 in the opposite direction, the roller assembly 320 is moved upward along the slider guides 328. For example, the hand crank 340 may be rotated in one direction (e.g., clockwise) until the IV bag 190 is empty, then hand crank 340 may be rotated in the opposite direction (e.g., counterclockwise) to move the roller assembly 320 back to the top position (e.g., starting position), whereupon the empty IV bag 190 may be removed from the IV bag wringer 300 and a full IV bag 190 placed into the IV bag wringer 300.

In aspects of the disclosure, the IV bag wringer 300 has an open frame configuration as shown in FIGS. 6 to 8. In aspects of the disclosure, the IV bag wringer 300 may include a enclosed housing having a front wall and a bottom wall such that the housing encloses some or all of the above-listed elements other than the hand crank 340. In aspects of the disclosure, such an enclosure may have a door or openable portion, providing for opening the IV bag wringer 300 to insert or place an IV bag 190 inside.

In aspects of the disclosure, the IV bag wringer 300 may include any suitable fastener to mount the IV bag wringer 300 to an IV pole (e.g., IV pole 46), a bed rail, a wall, a countertop, or any other suitable surface. For example, the fastener may be a bracket, a hangar, a hook, Velcro®, adhesive, and/or any other suitable fastener.

As shown in FIG. 9, an IV bag wringer 400 is provided according to aspects of the disclosure. IV bag wringer 400 may have some or almost all of the features of IV bag wringer 300, with a difference being that IV bag wringer 400 has a motor 440 coupled to one lead screw end 334 instead of the hand crank 340. For example, a lead screw end 334 may extend through the opening 335 of one lead screw mounting bracket 333, through an opening 317 in the side wall 316 and be received within a cavity or opening 442 of the motor 440.

In an actuated state, the motor 440 causes the lead screw 332 to rotate, causing the engaged gears 339 of the side screws 336 to rotate, and causing the threaded shafts 338 to rotate within the threaded openings 329 of the linear slide bearings 322 as well. Depending upon the direction of rotation of the motor 440, the linear slide bearings 322 are slidably moved up or down along the slider guides 328 based on the interaction between the threaded shafts 338 rotating within the threaded openings 329 of the linear slide bearings 322. The slidable movement of the linear slide bearings 322 causes the roller 324 to move in the same direction. In an unactuated state, the motor 440 remains static and thus the roller 324 remains in its current position.

In use, a fluid containing IV bag 190 may be placed within the IV bag wringer 400 where the IV bag 190 is disposed against the rear wall 312 in between the slider guides 328. For example, the top of the IV bag 190 may be placed or inserted between the rear wall 312 and the roller 324. With the IV bag 190 in position, the motor 440 may be operated (e.g., energized and rotating the coupled lead screw end 334), causing the lead screw 332 to rotate, in turn causing the side screws 336 to rotate. The rotation of the side screws 336 causes the linear slide bearings 322 to move downward along the slider guides 328, in turn causing the roller 324 to move downward. The downward movement of the roller 324 squeezes the IV bag 190 between the rear wall 312 and the roller 324, thus increasing the fluid flow rate out of the IV bag 190 over a gravity based flow rate alone. The flow rate can be increased by increasing the speed of the motor 440, thereby rotating the lead screw 332 faster to squeeze the IV bag 190 more quickly and/or forcefully.

In aspects of the disclosure, the gear ratio between the lead screw 332 and the gears 339 of the side screws 336 may be adjusted to increase or decrease the squeezing movement of the roller assembly 320. Thus, the IV bag wringer 300, 400 may be configured to work with different sized and/or shaped IV bags. In aspects of the disclosure, the IV bag wringer 300, 400 is reusable while the IV set 120 and/or the IV bag 190 is disposable. Accordingly, the IV bag wringer 300, 400 may be cleaned as necessary or per medical cleaning protocols.

In aspects of the disclosure, a pressure sensor assembly may be coupled to the output line 192 of the IV bag 190 in order to provide a real time in-line pressure measurement of the fluid flow from the IV bag 190. In aspects of the disclosure, a feedback loop may be coupled between the sensor assembly and the motor 440, thus providing for adjusting the speed of the motor 440 based on the real time pressure measurement from the sensor assembly. In aspects of the disclosure, the pressure sensor assembly may include or be coupled with a visual indicator to provide real time visual indication of the fluid flow pressure. For example, the visual indicator may visually show if the fluid flow pressure exceeds a predetermined limit (e.g., safe or acceptable limit).

In aspects of the disclosure, an IV bag may be inserted into an IV bag wringer (e.g., IV bag wringer 400), which may be set up and/or controlled by a controller. Thus, the desired flow rate of blood from the IV bag may be delivered through the IV tubing (e.g., IV tubing 160) into a patient by controlling the speed of the moving elements of the IV bag wringer.

In some embodiments according to the disclosure, an IV bag wringer comprises a frame defining a frame opening; and a plurality of roller assemblies disposed within the frame opening, each roller assembly comprising: a shaft coupled to the frame; and a roller coupled to the shaft, wherein two opposing rollers are positioned to receive an IV fluid bag in a gap between the rollers such that the IV bag wringer hangs on the IV fluid bag, and wherein the IV bag wringer is configured to move downward along the IV fluid bag based on a weight of the IV bag wringer such that the opposing rollers squeeze fluid out of the IV fluid bag at a rate greater than a gravity flow rate from the IV fluid bag alone.

In aspects of the disclosure, each roller assembly comprises: a first shaft end fixedly disposed within a first cavity in the frame; and a second shaft end fixedly disposed within a second cavity in the frame, wherein the roller is rotatably coupled to the shaft. In aspects of the disclosure, each roller assembly comprises: a first shaft end rotatably disposed within a first cavity in the frame; and a second shaft end rotatably disposed within a second cavity in the frame, wherein the roller is fixedly coupled to the shaft. In aspects of the disclosure, at least one roller assembly is adjustably coupled to the frame, the adjustably coupled roller assembly configured to adjust a size of the gap between the opposing rollers based on an adjusted position.

In aspects of the disclosure, one or more weights are coupled to the frame, wherein a total amount of weight of the one or more weights is configured to obtain a determined fluid flow rate from the IV fluid bag. In aspects of the disclosure, the one or more weights are integrally formed with the frame. In aspects of the disclosure, the one or more weights are fixedly coupled to the frame. In aspects of the disclosure, the one or more weights are removably coupled to the frame. In aspects of the disclosure, a restraining member is configured to hold the IV bag wringer in an unactuated state relative to the IV fluid bag.

In some embodiments according to the disclosure, an IV bag wringer comprises a housing comprising a rear wall, a top wall, a first side wall and an opposing second side wall; a roller assembly coupled to rear wall of the housing, the roller assembly comprising: a first linear slide bearing; a second linear slide bearing; a shaft coupled to the first and second linear slide bearings; and a roller coupled to the shaft; and a crank assembly coupled to the housing and to the first and second linear slide bearings, the crank assembly configured to slidably adjust a position of the first and second linear slide bearings relative to the rear wall, wherein a gap between the roller and the rear wall of the housing is configured to receive an IV fluid bag.

In aspects of the disclosure, a first slider guide is fixedly coupled to the rear wall of the housing, wherein the first linear slide bearing is slidably coupled to the first slider guide; and a second slider guide is fixedly coupled to the rear wall of the housing parallel to the first slider guide, wherein the second linear slide bearing is slidably coupled to the second slider guide. In aspects of the disclosure, a first side screw is rotatably coupled to a first threaded opening in the first linear slide bearing; and a second side screw is rotatably coupled to a second threaded opening in the second linear slide bearing, wherein rotation of the first and second side screws is configured to slidably move the first and second linear slide bearings along the first and second slider guides. In aspects of the disclosure, the first and second side screws are rotatably coupled to first and second side screw mounting brackets that are fixedly coupled to the top wall of the housing.

In aspects of the disclosure, the crank assembly comprises: a lead screw rotatably coupled to the first and second side walls of the housing, wherein a first portion of the lead screw is coupled to a first gear of the first side screw and a second portion of the lead screw is coupled to a second gear of the second side screw, wherein rotation of the lead screw is configured to cause corresponding rotation of the first and second side screws. In aspects of the disclosure, the lead screw is rotatably coupled to first and second lead screw mounting brackets that are fixedly coupled to the first and second side walls, respectively.

In aspects of the disclosure, a hand crank is fixedly coupled to the lead screw, the hand crank rotatably disposed at an external surface of the first side wall of the housing, wherein manual rotation of the hand crank is configured to cause the roller to move along the IV fluid bag to squeeze fluid out of the IV fluid bag at a rate greater than a full open gravity flow rate from the IV fluid bag alone, wherein a first speed of rotation of the hand crank corresponds to a first level of increase in the fluid flow rate from the IV fluid bag. In aspects of the disclosure, a motor fixedly coupled to the lead screw, the motor rotatably disposed at an external surface of the first side wall of the housing, wherein actuation of the motor is configured to cause the roller to move along the IV fluid bag to squeeze fluid out of the IV fluid bag at a rate greater than a full open gravity flow rate from the IV fluid bag alone, wherein a first speed of the motor corresponds to a first level of increase in the fluid flow rate from the IV fluid bag.

In some embodiments according to the disclosure, a method of operating an IV bag wringer comprises coupling an IV set to an IV fluid bag; hanging the IV fluid bag; setting a gravity based fluid flow rate of fluid from the IV fluid bag through the IV set; disposing a top portion of the IV fluid bag within the gap between the two opposing rollers of the IV bag wringer; hanging the IV bag wringer from the IV fluid bag; releasing the IV bag wringer such that a weight of the IV bag wringer moves the two opposing rollers downward along the IV fluid bag at a first speed; and squeezing the IV fluid bag between the two opposing rollers to cause an increased fluid flow rate from the IV fluid bag corresponding to the first speed.

In some embodiments according to the disclosure, a method of operating an IV bag wringer comprises coupling an IV set to an IV fluid bag; hanging the IV fluid bag; setting a gravity based fluid flow rate of fluid from the IV fluid bag through the IV set; disposing a top portion of the IV fluid bag within the gap between the roller and the rear wall of the IV bag wringer; rotating a hand crank coupled to the crank assembly; moving the roller downward along the IV fluid bag at a first speed based on a rate of rotation of the hand crank; and squeezing the IV fluid bag between the roller and the rear wall to cause an increased fluid flow rate from the IV fluid bag corresponding to the first speed.

In some embodiments according to the disclosure, a method of operating an IV bag wringer comprises coupling an IV set to an IV fluid bag; hanging the IV fluid bag; setting a gravity based fluid flow rate of fluid from the IV fluid bag through the IV set; disposing a top portion of the IV fluid bag within the gap between the roller and the rear wall of the IV bag wringer; actuating a motor coupled to the crank assembly; moving the roller downward along the IV fluid bag at a first speed based on the speed of the motor; and squeezing the IV fluid bag between the roller and the rear wall to cause an increased fluid flow rate from the IV fluid bag corresponding to the first speed.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) bag wringer, comprising:
a frame defining a frame opening; and
a plurality of roller assemblies disposed within the frame opening, each roller assembly comprising:
a shaft coupled to the frame; and
a roller coupled to the shaft,
wherein two opposing rollers are positioned to receive an IV fluid bag in a gap between the rollers such that the IV bag wringer hangs on the IV fluid bag, and
wherein the IV bag wringer is configured to move downward along the IV fluid bag based on a weight of the IV bag wringer such that the opposing rollers squeeze fluid out of the IV fluid bag at a rate greater than a gravity flow rate from the IV fluid bag alone.

2. The IV bag wringer of claim 1, wherein each roller assembly comprises:
a first shaft end fixedly disposed within a first cavity in the frame; and
a second shaft end fixedly disposed within a second cavity in the frame,
wherein the roller is rotatably coupled to the shaft.

3. The IV bag wringer of claim 1, wherein each roller assembly comprises:
a first shaft end rotatably disposed within a first cavity in the frame; and
a second shaft end rotatably disposed within a second cavity in the frame,
wherein the roller is fixedly coupled to the shaft.

4. The IV bag wringer of claim 1, wherein at least one roller assembly is adjustably coupled to the frame, the adjustably coupled roller assembly configured to adjust a size of the gap between the opposing rollers based on an adjusted position.

5. The IV bag wringer of claim 1, further comprising one or more weights coupled to the frame, wherein a total amount of weight of the one or more weights is configured to obtain a determined fluid flow rate from the IV fluid bag.

6. The IV bag wringer of claim 5, wherein the one or more weights are integrally formed with the frame.

7. The IV bag wringer of claim 5, wherein the one or more weights are fixedly coupled to the frame.

8. The IV bag wringer of claim 5, wherein the one or more weights are removably coupled to the frame.

9. The IV bag wringer of claim 1, further comprising a restraining member configured to hold the IV bag wringer in an unactuated state relative to the IV fluid bag.

10. An intravenous (IV) bag wringer, comprising:
a housing comprising a rear wall, a top wall, a first side wall and an opposing second side wall;
a roller assembly coupled to rear wall of the housing, the roller assembly comprising:
a first linear slide bearing;
a second linear slide bearing;
a shaft coupled to the first and second linear slide bearings; and
a roller coupled to the shaft; and
a crank assembly coupled to the housing and to the first and second linear slide bearings, the crank assembly configured to slidably adjust a position of the first and second linear slide bearings relative to the rear wall,
wherein a gap between the roller and the rear wall of the housing is configured to receive an IV fluid bag.

11. The IV bag wringer of claim 10, further comprising:
a first slider guide fixedly coupled to the rear wall of the housing, wherein the first linear slide bearing is slidably coupled to the first slider guide; and
a second slider guide fixedly coupled to the rear wall of the housing parallel to the first slider guide, wherein the second linear slide bearing is slidably coupled to the second slider guide.

12. The IV bag wringer of claim 11, further comprising:
a first side screw rotatably coupled to a first threaded opening in the first linear slide bearing; and
a second side screw rotatably coupled to a second threaded opening in the second linear slide bearing,
wherein rotation of the first and second side screws is configured to slidably move the first and second linear slide bearings along the first and second slider guides.

13. The IV bag wringer of claim 12, wherein the first and second side screws are rotatably coupled to first and second side screw mounting brackets that are fixedly coupled to the top wall of the housing.

14. The IV bag wringer of claim 12, wherein the crank assembly comprises:
a lead screw rotatably coupled to the first and second side walls of the housing, wherein a first portion of the lead screw is coupled to a first gear of the first side screw and a second portion of the lead screw is coupled to a second gear of the second side screw, wherein rotation of the lead screw is configured to cause corresponding rotation of the first and second side screws.

15. The IV bag wringer of claim 14, wherein the lead screw is rotatably coupled to first and second lead screw mounting brackets that are fixedly coupled to the first and second side walls, respectively.

16. The IV bag wringer of claim 14, further comprising:
a hand crank fixedly coupled to the lead screw, the hand crank rotatably disposed at an external surface of the first side wall of the housing,
wherein manual rotation of the hand crank is configured to cause the roller to move along the IV fluid bag to squeeze fluid out of the IV fluid bag at a rate greater than a full open gravity flow rate from the IV fluid bag alone,
wherein a first speed of rotation of the hand crank corresponds to a first level of increase in the fluid flow rate from the IV fluid bag.

17. The IV bag wringer of claim 14, further comprising:
a motor fixedly coupled to the lead screw, the motor rotatably disposed at an external surface of the first side wall of the housing,
wherein actuation of the motor is configured to cause the roller to move along the IV fluid bag to squeeze fluid out of the IV fluid bag at a rate greater than a full open gravity flow rate from the IV fluid bag alone,
wherein a first speed of the motor corresponds to a first level of increase in the fluid flow rate from the IV fluid bag.

18. A method of operating the IV bag wringer of claim 1, the method comprising:
coupling an IV set to an IV fluid bag;
hanging the IV fluid bag;
setting a gravity based fluid flow rate of fluid from the IV fluid bag through the IV set;
disposing a top portion of the IV fluid bag within the gap between the two opposing rollers of the IV bag wringer;
hanging the IV bag wringer from the IV fluid bag;
releasing the IV bag wringer such that a weight of the IV bag wringer moves the two opposing rollers downward along the IV fluid bag at a first speed; and
squeezing the IV fluid bag between the two opposing rollers to cause an increased fluid flow rate from the IV fluid bag corresponding to the first speed.

19. A method of operating the IV bag wringer of claim 10, the method comprising:
coupling an IV set to an IV fluid bag;
hanging the IV fluid bag;
setting a gravity based fluid flow rate of fluid from the IV fluid bag through the IV set;
disposing a top portion of the IV fluid bag within the gap between the roller and the rear wall of the IV bag wringer;
rotating a hand crank coupled to the crank assembly;
moving the roller downward along the IV fluid bag at a first speed based on a rate of rotation of the hand crank; and
squeezing the IV fluid bag between the roller and the rear wall to cause an increased fluid flow rate from the IV fluid bag corresponding to the first speed.

20. A method of operating the IV bag wringer of claim 10, the method comprising:
coupling an IV set to an IV fluid bag;
hanging the IV fluid bag;
setting a gravity based fluid flow rate of fluid from the IV fluid bag through the IV set;
disposing a top portion of the IV fluid bag within the gap between the roller and the rear wall of the IV bag wringer;
actuating a motor coupled to the crank assembly;
moving the roller downward along the IV fluid bag at a first speed based on the speed of the motor; and
squeezing the IV fluid bag between the roller and the rear wall to cause an increased fluid flow rate from the IV fluid bag corresponding to the first speed.
